(19) United States
(12) Patent Application Publication
(10) Pub. No.: US 2026/0306239 A1
(43) Pub. Date: Oct. 8, 2026

(54) VENOUS EMBOLIC PROTECTION FILTER

(71) Applicant: KONINKLIJKE PHILIPS N.V., EINDHOVEN (NL)

(72) Inventor: Chad PERRIN, MAPLE GROVE, MN (US)

(21) Appl. No.: 19/481,978

(22) PCT Filed: Apr. 30, 2024
(86) PCT No.: PCT/EP2024/061817

§ 371 Date: Nov. 6, 2025

Related U.S. Application Data
(60) Provisional application No. 63/465,271, filed on May 10, 2023.

(51) Int. Cl. A61F 2/01; A61B 17/32; A61B 18/24; A61F 2/95; A61M 25/09; A61M 25/10

(52) U.S. Cl. CPC A61F 2/013; A61F 2/0108; A61F 2/011; A61B 17/320016; A61B 18/24; A61F 2002/016; A61F 2/95; A61F 2210/0014; A61F 2230/0065; A61F 2250/001; A61M 25/09; A61M 25/104

(57) ABSTRACT

An intravascular device (10) includes a rail catheter (12) having a lumen (13) sized to receive an associated guidewire (14); a self-expanding strainer (16) having an open end (40) attached to a distal end of the rail catheter by a plurality of tethers (18) and a closed end (42) opposite the open end, the closed end being closed except for a guidewire passthrough sized to receive the associated guidewire; and a sheath (26) with a lumen (28) sized to receive the rail catheter and having a distal end configured to receive the self-expanding strainer compressed into the distal end of the sheath.

### FIELD

The following relates generally to the catheter arts, intravascular therapy arts, lesion treatment arts, and related arts.

### BACKGROUND

Intravascular procedures such as thrombectomy, angioplasty, stent delivery, and so forth are used to treat vascular occlusions (e.g., clots, plaque, or other types of intravascular build-ups). A risk with such procedures is that portions of the occlusion can be dislodged during the procedure, resulting in debris that enters the bloodstream and potentially can produce secondary clinical effects such as embolization. This problem can be combatted using aspiration, but since aspiration flow is counter to the blood flow, the aspiration may fail to capture some debris. Filters can be incorporated into the treatment device to capture the debris, but the filter increases complexity of the treatment device and can interfere with performing the intervascular therapy using the treatment device. In yet another approach, an embolic filter can serve as the therapy device, and is drawn along the blood vessel in a retraction type of motion opposite the direction of blood flow. Here the filter is itself the therapy device, and the debris produced by engagement of the retracting filter with the occlusion is captured by the retracting filter. However, this approach is not suited to provide embolic protection for another type of intravascular procedure performed with a different instrument, such as thrombectomy performed using a mechanical or laser cutter, or angioplasty performed using an expanding balloon.

The following discloses certain improvements to overcome these problems and others.

### SUMMARY

In some embodiments disclosed herein, an intravascular device includes a rail catheter having a lumen sized to receive an associated guidewire; a self-expanding strainer having an open end attached to a distal end of the rail catheter by a plurality of tethers and a closed end opposite the open end, the closed end being closed except for a guidewire passthrough sized to receive the associated guidewire; and a sheath with a lumen sized to receive the rail catheter and having a distal end configured to receive the self-expanding strainer compressed into the distal end of the sheath.

In some embodiments disclosed herein, an intravascular device includes a rail catheter having a lumen sized to receive an associated guidewire; and a self-expanding strainer having an open end attached to a distal end of the rail catheter by a plurality of tethers and a closed end configured to be secured around the associated guidewire.

In some embodiments disclosed herein, an intravascular therapy method includes inserting a guidewire into a blood vessel and past an occlusion of the blood vessel; delivering a self-expanding strainer having an open end tethered to a rail catheter by a plurality of tethers into the blood vessel and past the occlusion by passing the rail catheter along the guidewire, wherein during the delivering the self-expanding strainer is compressed inside a sheath surrounding the rail catheter and the self-expanding strainer; withdrawing the sheath to deploy the self-expanding strainer whereby the self-expanding strainer expands inside the blood vessel at a location past the occlusion of the blood vessel; after the withdrawing, delivering a treatment device to the occlusion by passing a treatment catheter over the rail catheter; and performing treatment of the occlusion with the treatment device.

One advantage resides in providing an embolic protection filter deployed downstream of venous blood flow away from a treatment site, thus reducing the potential for the filter to interfere with treatment performed at the treatment site.

Another advantage resides in controlling a filter of an intravascular device with fiber tethers that prevent accidental movement.

Another advantage resides in providing an intravascular device to improve patient safety by capturing more thrombus material in the bloodstream.

Another advantage resides in providing a filter of an intravascular device to allow for aspiration within the filter volume during an intravascular therapy procedure.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an intravascular therapy device in accordance with the present disclosure.

FIGS. 2, 3, 4, and 5 diagrammatically show an embolic protection filter assembly at different stages of use, including: FIG. 2 showing the filter assembly in the delivery configuration with the self-expanding strainer (also referred to as a ‘filter’) packed inside a delivery sheath; FIG. 3 showing the deployed configuration with the delivery sheath withdrawn and the self-expanding strainer in its expanded state; FIG. 4 shows the deployed configuration with the treatment or therapy device or catheter delivered along the rail catheter to the treatment site; and FIG. 5 showing a retrieval sheath being deployed at a point as the self-expanding strainer is beginning to enter the distal end of the retrieval sheath.

FIG. 6 diagrammatically shows another embodiment of an embolic protection filter assembly in isolation view, showing the deployed configuration with the delivery sheath withdrawn and the self-expanding strainer in its expanded state.

FIG. 7 diagrammatically illustrates an exemplary flowchart of operations performed with the device of FIG. 1.

### DETAILED DESCRIPTION

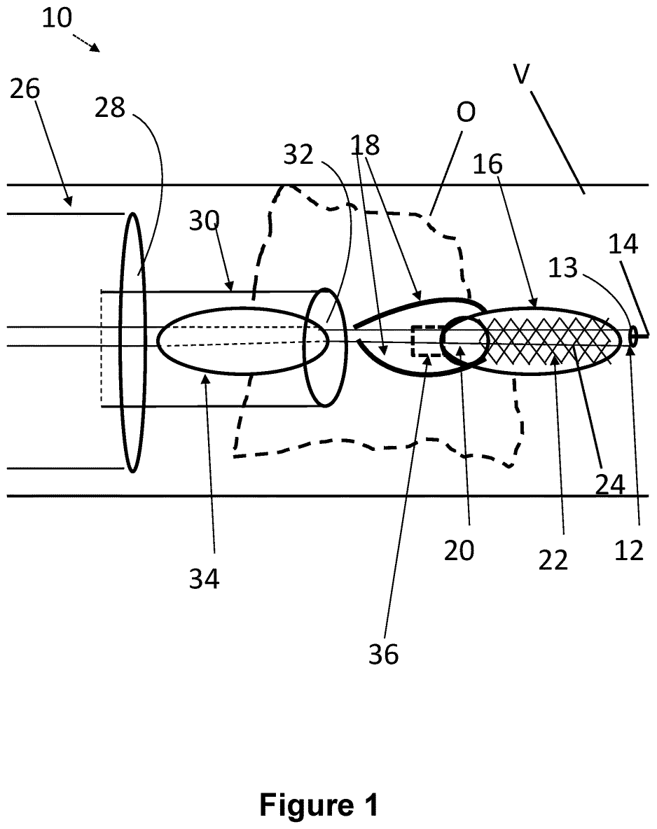

With reference to FIGS. 1-4, a protective filter is disclosed, which provides for capturing fragments generated by thrombectomy, aspiration, angioplasty, stent delivery, or other intravascular procedures. The filter is in the form of a self-expanding strainer whose mouth is secured by multiple tethers (e.g., polymer monofilaments, or sutures or other fabric or polymer threads) to the distal end of a filter rail catheter. For delivery, the self-expanding strainer is compacted inside the rail catheter. The filter rail catheter (with contained strainer) and a surrounding deployment sheath are delivered over a guidewire to place the filter at a location downstream of the treatment site. The deployment sheath is then withdrawn to release the filter, which is self-expanding. After withdrawing the delivery sheath, the filter rail catheter remains in place, with the expanded filter anchored to the filter rail catheter by the tethers.

A treatment catheter having a treatment device at its distal end is then delivered along the filter rail catheter (within which the guidewire is disposed) to place the treatment device at the treatment site (with the filter remaining downstream of the treatment site). For compatibility with standard treatment catheters, the filter rail catheter may be of small diameter, e.g., 35-thousandths diameter in some cases. As the filter is packed in the deployment sheath for delivery, rather than in the filter rail catheter, there is advantageously no practical limitation on the diameter of the filter rail catheter other than that it has a lumen diameter sufficient to receive the 18-thousandths guidewire.

The treatment device can be a mechanical cutter, a laser ablator, an angioplasty balloon, or even a stent to be delivered. During treatment, fragments of the clot or other occlusion may break loose, and these are captured by the downstream filter.

After treatment, the treatment catheter is withdrawn along the filter rail catheter, and a retrieval sheath is inserted along the filter rail catheter until the end of the retrieval sheath captures the tethers. This advantageously functions to close the mouth of the strainer. Continued insertion then captures the now-closed filter in the lumen of the retrieval sheath. Counter-tension may be applied to the filter rail catheter to facilitate the filter mouth closing and capture process. With the filter captured inside the lumen of the retrieval sheath, the combination of the filter rail catheter and retrieval sheaths are withdrawn along the guidewire. The guidewire may optionally be thereafter used for further intravascular tasks such as delivering an intravascular ultrasound (IVUS) device to inspect the treatment site. Finally, the guidewire is removed.

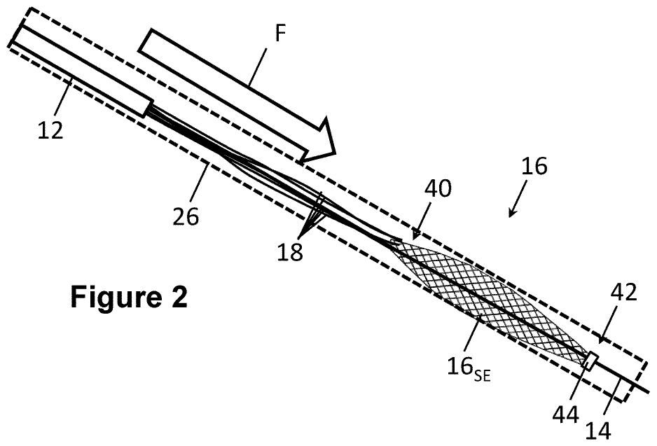

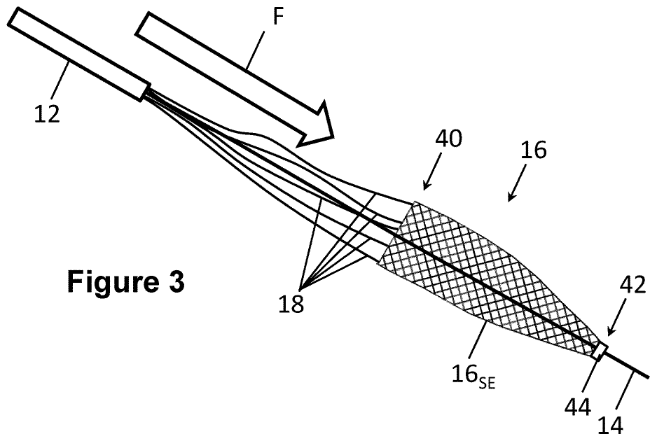

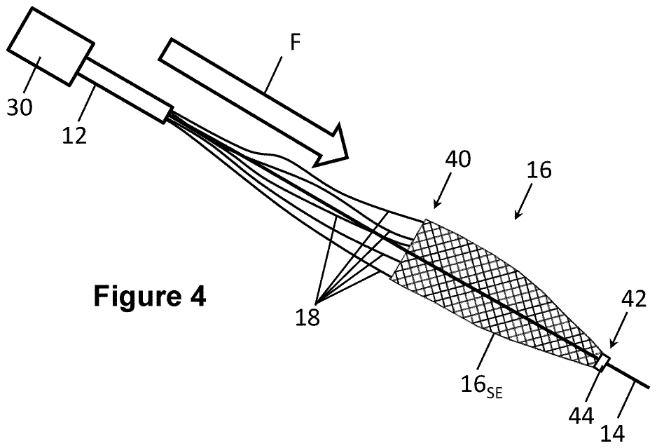

The filter is self-expanding. Some suitable constructions include a self-expanding nitinol strainer (see FIGS. 2 and 3), or a strainer in the form of a fabric or polymer basket (i.e., strainer mesh) with a self-expanding nitinol ring secured to the mouth of the fabric or polymer basket (see FIG. 4). When deployed, the open end of the strainer faces the treatment site (i.e., faces upstream), and the tethers connect with the open end. The end of the filter opposite the open end is a closed end, and may suitably be closed by a (non-expanding) small-diameter ring through which the guidewire passes.

The design advantageously places the filter downstream of the treatment site, so that the filter does not interfere with the treatment. The treatment device should not be delivered past the end of the filter rail catheter, since otherwise the tethers of the filter could get entangled with the treatment device (or, in the case of a cutter, the tethers could be cut by the cutter). In some embodiments, the filter rail catheter includes a nub, boss, or other protrusion that acts as a stop to prevent the treatment device from being pushed past the nub, boss, or other protrusion.

The pores of the filter should be sufficiently large to enable packing into the delivery/retrieval sheaths, and to ensure that the filter does not introduce too much blood flow resistance. In some embodiments, the filter may have a pore size of at least 0.25 mm, and more typically at least 0.75 mm. These dimensions are for the expanded filter. If the filter employs crossing helical wires, then the pores will be quadrilateral in shape and may be measured corner-to-corner along the direction of the guidewire.

The disclose device can be used for intravascular procedures performed in the venous system, such as thrombectomies. The filter could also be used in intravascular procedures performed in the arterial system. The balance of pore size versus packability/flow resistance can be more challenging in this case, as pore sizes on the order of 100-200 microns are typically preferred in arterial procedures due to the higher risk posed by smaller fragments passing though the arterial system.

With reference to FIG. 1, an illustrative intravascular device 10 is diagrammatically shown. As shown in FIG. 1, the intravascular device 10 is insertable into a blood vessel V for treating an occlusion O (or a clot, or a lesion, and so forth) in the blood vessel V. The intravascular therapy device 10 includes, for example, a rail catheter 12 having a lumen 13 sized to receive a guidewire 14.

A self-expanding strainer 16 is configured to collect fragments from the occlusion O during an intravascular therapy procedure. To do so, the self-expanding strainer 16 has an open end attached to a distal end of the rail catheter by a plurality of tethers 18 (two of which are shown in FIG. 1), and a closed end opposite the open end that is configured to be secured around the guidewire 14. The closed end is closed except for a guidewire passthrough in the lumen 13 sized to receive the guidewire 14. For example, the self-expanding strainer 16 can include at least one self-expanding ring 20 (one of which is shown in FIG. 1) disposed at the open end of the self-expanding strainer 16. A basket 22 has an open end secured to the self-expanding ring(s) 20 and further forms the closed end of the self-expanding strainer 16. In some embodiments, the self-expanding strainer 16 and/or the ring(s) 20 comprise nitinol (i.e., are self-expanding), and the basket 22 comprises a fabric and/or a polymer (i.e., is not self-expanding). The guidewire passthrough of the closed end of the self-expanding strainer 16 (i.e., where the backet 22 is disposed) has a diameter of 0.15 centimeter or less and the open end of the self-expanding strainer has a diameter of at least 0.5 centimeter. FIG. 1 shows the self-expanding strainer 16 in an expanded state. Pores 24 of the self-expanding strainer 16 have a diameter of at least 0.5 mm when the self-expanding strainer is expanded.

FIG. 1 also shows a sheath 26 with a lumen 28 sized to receive the rail catheter 12, and has a distal end configured to receive the self-expanding strainer 16 compressed into the distal end of the sheath 26. The sheath 26 may be a delivery sheath or a retrieval sheath, depending on whether it is being used to deliver the filter prior to intravascular treatment or therapy or to remove it after the intravascular treatment or therapy. FIG. 1 further shows a treatment or therapy catheter 30 having a guidewire lumen 32. The rail catheter 12 is sized to be received by the guidewire lumen 32 of the treatment catheter 30. Advantageously, the rail catheter 12 has a small enough diameter to fit inside guidewire lumen 32 of the treatment catheter 30. While FIG. 1 shows both the treatment or therapy catheter 30 and the delivery or retrieval sheath 26, it should be noted that in some typical procedure workflows, both the sheath 26 and treatment or therapy catheter 30 would not be deployed along the guidewire 14 at the same time.

The treatment catheter 30 includes a treatment device 34 disposed at a distal end of the treatment catheter 30. The treatment device 34 is depicted in FIG. 1 as an angioplasty balloon but can be any suitable intravascular treatment device (e.g., a mechanical cutter, a laser cutter, a stent, and so forth). The treatment catheter 30 is configured to be delivered over the rail catheter 12 as the rail catheter 12 passes through the guidewire lumen 32 to deliver the treatment device 34 to the occlusion O. In some embodiments, the rail catheter 12 includes a protrusion 36 configured to prevent the treatment device 34 from being delivered past the protrusion 36.

With reference now to FIGS. 2-5, a first illustrative embodiment of the intravascular device 10 is shown in more detail and is presented in a workflow sequence. In this sequence, it is to be understood that the device is located intravascularly, that is, with at least the illustrated distal end positioned inside a blood vessel of a patient. FIGS. 2-5 include an arrow F indicating the direction of blood flow.

FIG. 2 shows the intravascular device 10 in the delivery configuration. In this configuration, the self-expanding strainer 16 is packed inside a delivery sheath 26 (shown in phantom as indicated by dashed lines). In this embodiment, the self-expanding strainer 16 is suitably a self-expanding strainer 16SE such as a self-expanding nitinol strainer 16SE that is fabricated in the expanded configuration under conditions such that the nitinol is a memory material that returns the nitinol strainer 16SE to its initially set expanded state in the absence of external forces. As shown in FIG. 2, however, the nitinol strainer 16SE is compacted inside a lumen of the distal end of the delivery sheath 26 to facilitate delivery of the sheath 26 with the filter assembly (including the nitinol strainer 16SE tethered to the rail catheter 12 by the plurality of tethers 18) disposed inside the lumen of the sheath 26. More particularly, the guidewire 14 is first inserted into the patient's vasculature and pushed to move the self-expanding strainer 16 past the occlusion or other treatment site. This delivery is typically done under image guidance, e.g., with X-ray imaging to assist the surgeon in the insertion process. Notably, in the compacted configuration with the self-expanding strainer 16 compacted inside the sheath 26, the open end 40 of the self-expanding strainer 16 is compacted into at least a partially closed position. The closed end 42 of the self-expanding strainer 16 is opposite the open end 40. The closed end 42 is closed (even in the expanded state, see FIG. 3), except for a guidewire passthrough sized to receive the guidewire 14. In some embodiments, a ferrule 44 is secured at the closed end 42 to reinforce and define the passthrough and provide a smooth surface for traversal along the guidewire 14.

FIG. 3 shows the deployed configuration. To obtain this configuration, the delivery sheath 26 shown in FIG. 2 is removed (and hence is not shown in FIG. 3). With the constraint of the sheath 26 removed, the self-expanding strainer 16 expands to its expanded state, in which the open end 40 is fully open. (Although, the expansion of the open end 40 could be constrained by the surrounding blood vessel, not shown in FIGS. 2-4). However, the open end 40 of the expanded strainer 16 remains tethered to the distal end of the rail catheter 12 by the tethers 18. The expanded strainer 16 is located downstream of the treatment site (i.e., downstream relative to the direction F of blood flow). Hence, the expanded strainer 16 is positioned to capture any debris from the treatment site that may flow downstream from the treatment site. The strainer 16 will retain any debris that is larger than the size of the pores 24 of the strainer (see FIG. 1). The guidewire passthrough at the closed end 42 should also be small enough to prevent debris of the size intended to be strained by the strainer 16 from escaping via the guidewire passthrough.

FIG. 4 shows the deployed configuration of FIG. 3, but now with the treatment or therapy device or catheter 30 delivered along the rail catheter 12 to the treatment site (which, again, is upstream of the strainer 16 where “upstream” is referenced to the blood flow F). Notably, the rail catheter 12 should have an outer diameter (OD) that is small enough so that the rail catheter can be received into the guidewire lumen of the treatment catheter 30. For typical guidewire diameters (and consequently typical guidewire lumen inner diameters), the OD of the rail catheter 12 may be 0.15 centimeter or less in some embodiments. On the other hand, the open end 40 of the self-expanding strainer 40 has a larger diameter, for example at least 0.5 centimeter in some embodiments, to provide a large opening to capture any debris of the occlusion that may be produced by the treatment or therapy performed using the upstream treatment or therapy device or catheter 30.

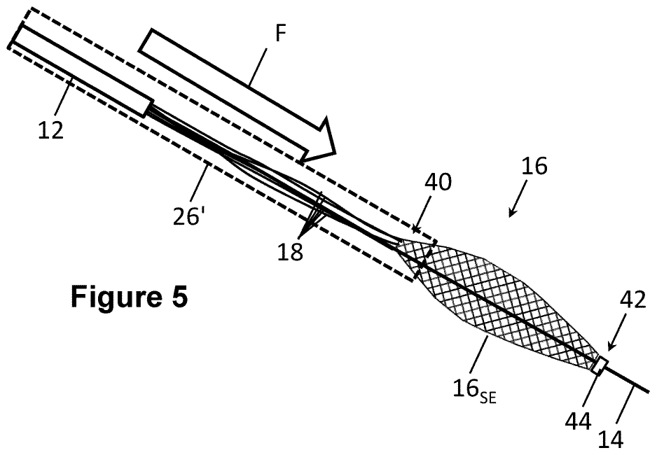

FIG. 5 shows the next stage of the workflow. After the treatment or therapy is complete, a retrieval sheath 26′ (which may be the same sheath as the delivery sheath 26, or may be different sheaths to ensure sterility) is deployed by being inserted along the rail catheter 12 to capture the self-expanding strainer 16. FIG. 5 depicts the point in time where the distal end of the advancing retrieval sheath 26′ is beginning to capture the open end 40 of the strainer 16. As shown, the tethers 18 are first drawn into the retrieval sheath 26′—this draws the tethers 18 together to advantageously automatically close the open end 40 of the strainer 16 as it approaches the distal end of the retrieval sheath 26′. This ensures that the self-expanding strainer 16 is safely and easily captured by the advancing retrieval sheath 26′. After the strainer 16 is fully captured into (i.e., compacted into) the distal end of the retrieval sheath 26′, it and the rail catheter 12 can be withdrawn together along the guidewire 14, after which the guidewire 14 is removed to complete the intravascular procedure.

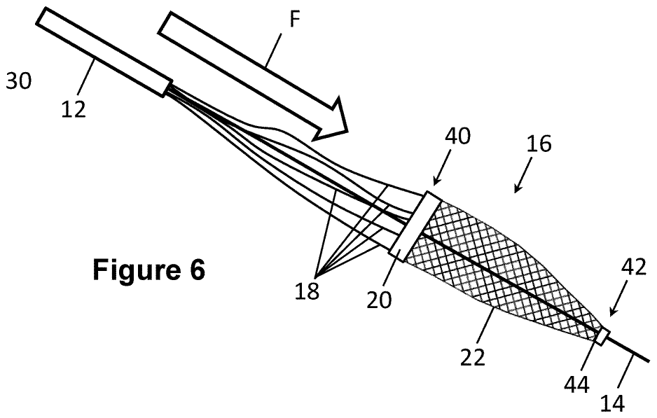

FIG. 6 diagrammatically shows a second embodiment, depicted in the deployed configuration analogous to that shown in FIG. 3 for the first embodiment. This embodiment differs in that the self-expanding strainer 16 is constructed differently. In the embodiment of FIG. 6, the self-expanding strainer 16 includes at least one self-expanding ring 20 (one of which is shown in FIG. 6) disposed at the open end 40 of the self-expanding strainer 16. A basket 22 has an open end coinciding with the open end 40 which is secured to the self-expanding ring(s) 20, and further forms the closed end 42 of the self-expanding strainer 16. The closed end may again optionally be reinforced by the ferrule 44. In some embodiments, the self-expanding ring(s) 20 comprise nitinol (i.e., are self-expanding), and the basket 22 comprises a fabric and/or a polymer (i.e., is not self-expanding).

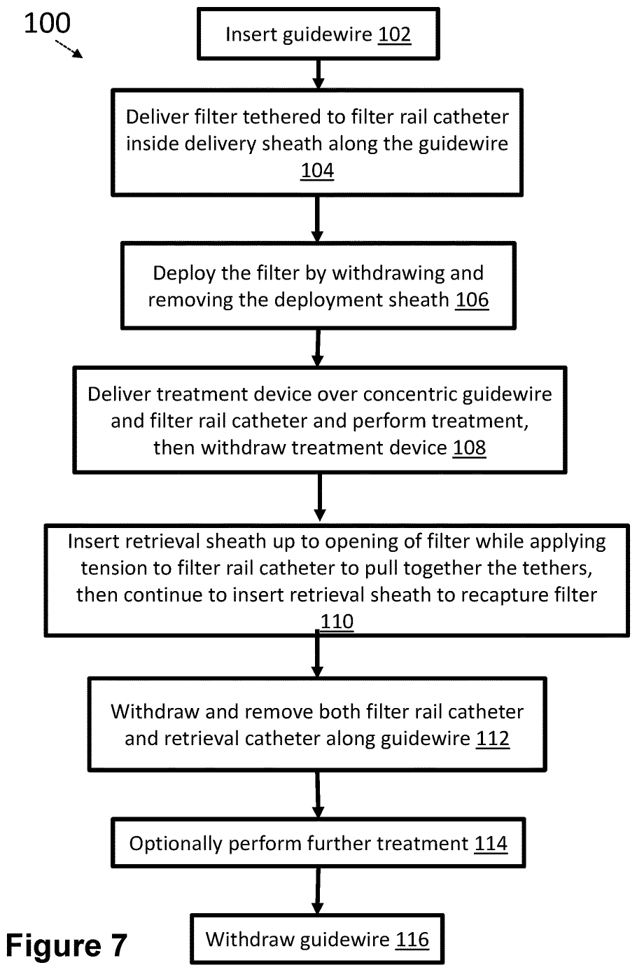

The device 10 can be used in an intravascular therapy method 100 to treat the occlusion O. FIG. 7 shows an exemplary operation of the method 100 as a flow chart. At an operation 102, the guidewire 14 is inserted into the blood vessel V and past the occlusion O (i.e., downstream with respect to the blood flow through the blood vessel V). At an operation 104, the self-expanding strainer 16 is inserted into the blood vessel V along the guidewire 14 and past the occlusion O by passing the rail catheter 12 and delivery catheter 26 along the guidewire 14, thereby reaching the state shown in FIG. 2. During the delivery operation 104, the self-expanding strainer 16 is compressed inside the sheath 26 surrounding the rail catheter 12 and the self-expanding strainer 14. At an operation 106, the sheath 26 is withdrawn to deploy the self-expanding strainer 16, thereby reaching the state shown in FIG. 3. The self-expanding strainer 14 expands inside the blood vessel V at a location past the occlusion O of the blood vessel V. At an operation 108, after the withdrawing of the sheath 26, the treatment device 34 is delivered to the occlusion O by passing the treatment catheter 32 over the rail catheter 12, thereby reaching the state shown in FIG. 4. Treatment of the occlusion O is performed with the treatment device 34. The treatment device 34 is then withdrawn from the blood vessel V.

At an operation 110, a retrieval catheter 26′ is inserted into the blood vessel V over the rail catheter 12 to compress the self-expanding strainer 16 into the retrieval catheter 12. The beginning stage of this compression is shown in FIG. 5. At an operation 112, the retrieval catheter and the rail catheter 12 are withdrawn from the blood vessel V with the self-expanding strainer 16 compressed in the retrieval catheter from the blood vessel V. At an optional operation 114, further treatment of the occlusion O is performed with the treatment device 34. At an operation 114, the guidewire 14 is withdrawn from the blood vessel V.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An intravascular device comprising:
a rail catheter having a lumen sized to receive an associated guidewire
a self-expanding strainer having an open end attached to a distal end of the rail catheter by a plurality of tethers and a closed end opposite the open end, the closed end being closed except for a guidewire passthrough sized to receive the associated guidewire; and
a sheath with a lumen sized to receive the rail catheter and having a distal end configured to receive the self-expanding strainer compressed into the distal end of the sheath.

2. The intravascular device of claim 1, further including:
a treatment catheter having a guidewire lumen and further having a treatment device disposed at a distal end of the treatment catheter,
wherein the rail catheter is sized to be received by the guidewire lumen of the treatment catheter, and the treatment catheter is configured to be delivered over the rail catheter passing through the guidewire lumen to deliver the treatment device to an associated blood vessel occlusion.

3. The intravascular device of claim 2, wherein the rail catheter includes a protrusion configured to prevent the treatment device from being delivered past the protrusion.

4. The intravascular device of claim 2, wherein the treatment device comprises one of a mechanical cutter, a laser cutter, an angioplasty balloon, or a stent.

5. The intravascular device of claim 1, wherein the self-expanding strainer comprises nitinol.

6. The intravascular device of claim 1, wherein the self-expanding strainer comprises:
at least one self-expanding ring disposed at the open end of the self-expanding strainer; and
a fabric and/or polymer basket having an open end secured to the at least one self-expanding ring and further forming the closed end of the self-expanding strainer.

7. The intravascular device of claim 6, wherein the fabric and/or polymer basket is not self-expanding.

8. The intravascular device of claim 6, wherein the at least one self-expanding ring comprises at least one self-expanding nitinol ring.

9. The intravascular device of claim 1, wherein a guidewire passthrough of the closed end of the self-expanding strainer has a diameter of 0.15 centimeter or less and the open end of the self-expanding strainer has a diameter of at least 0.5 centimeter.

10. The vascular therapy device of claim 1, wherein pores of the self-expanding strainer have a diameter of at least 0.5 mm when the self-expanding strainer is expanded.

11. An intravascular device comprising:
a rail catheter having a lumen sized to receive an associated guidewire and
a self-expanding strainer having an open end attached to a distal end of the rail catheter by a plurality of tethers and a closed end configured to be secured around the associated guidewire.

12. The intravascular device of claim 11, further comprising:
a sheath with a lumen sized to receive the rail catheter and having a distal end configured to receive the self-expanding strainer compressed into the distal end of the sheath; and
a treatment catheter having a treatment device disposed at an end thereof;
wherein the treatment catheter is configured to deliver the treatment device to an intravascular treatment site over the rail catheter.

13. The intravascular device of claim 12, wherein the rail catheter is sized to be received by a guidewire lumen of the treatment catheter and the treatment catheter is configured to be delivered over the rail catheter passing through the guidewire lumen to deliver the treatment device to an associated blood vessel occlusion.

14. The intravascular device of claim 12, wherein the treatment device comprises one of a mechanical cutter, a laser cutter, an angioplasty balloon, or a stent.

15. The intravascular device of claim 12, wherein the self-expanding strainer comprises nitinol.

16. The intravascular device of claim 11, wherein the self-expanding strainer comprises:
at least one self-expanding ring disposed at the open end of the self-expanding strainer; and
a fabric and/or polymer basket having an open end secured to the at least one self-expanding ring and further forming the closed end of the self-expanding strainer.

17. The intravascular device of claim 16, wherein the fabric and/or polymer basket is not self-expanding.

18. The intravascular device of claim 16, wherein the at least one self-expanding ring comprises at least one self-expanding nitinol ring.

19. An intravascular therapy method comprising:
inserting a guidewire into a blood vessel and past an occlusion of the blood vessel;
delivering a self-expanding strainer having an open end tethered to a rail catheter by a plurality of tethers into the blood vessel and past the occlusion by passing the rail catheter along the guidewire, wherein during the delivering the self-expanding strainer is compressed inside a sheath surrounding the rail catheter and the self-expanding strainer;
withdrawing the sheath to deploy the self-expanding strainer whereby the self-expanding strainer expands inside the blood vessel at a location past the occlusion of the blood vessel;
after the withdrawing, delivering a treatment device to the occlusion by passing a treatment catheter over the rail catheter; and
performing treatment of the occlusion with the treatment device.

20. The intravascular therapy method of claim 19, further comprising:
withdrawing the treatment device from the blood vessel;
inserting a retrieval catheter into the blood vessel over the rail catheter and compressing the self-expanding strainer into the retrieval catheter;
withdrawing the retrieval catheter and the rail catheter with the self-expanding strainer compressed in the retrieval catheter from the blood vessel; and
withdrawing the guidewire.
